(19) United States
(12) Patent Application Publication
(10) Pub. No.: US 2016/0030079 A1
(43) Pub. Date: Feb. 4, 2016

(54) CANNULA ASSEMBLY

(71) Applicant: Patrick Cohen, Maytar (IL)

(72) Inventor: Patrick Cohen, Maytar (IL)

(21) Appl. No.: 14/449,515

(22) Filed: Aug. 1, 2014

(51) Int. Cl. A61B 17/34; A61M 5/158

(52) U.S. Cl. CPC A61B 17/3421; A61B 17/3403; A61M 5/158; A61M 2240/00; A61B 2017/3454; A61M 2005/1587

(57) ABSTRACT

The invention provides an improved cannula assembly comprising a first cannula having a first length and first inner diameter, the first cannula having at least a flexible distal portion; a trochar having a second length and second diameter, the trochar having a sharp distal position capable of puncturing through skin and one or more blood vessels of a patient and sized such that the trochar may be inserted within the first cannula and extend beyond the flexible distal portion thereof; and at least one flexible second cannula for insertion in the first cannula, each second flexible cannula sized to extend beyond the distal portion of the first cannula for a predetermined length.

### FIELD OF THE INVENTION

This invention related to medical devices, and more specifically, to cannula assemblies for transferring a fluid into or out of a body cavity, such as a blood vessel.

### BACKGROUND OF THE INVENTION

Many medical procedures require access to a body cavity, such as a blood vessel, in order to deliver substances to the cavity or to withdraw fluids from the cavity. For example, a vein may be accessed in order to administer fluids and drugs, or for drawing blood for analyses. Accessing a body cavity, such as a blood vessel, often involves introducing a catheter onto a cannula and pushing the cannula tip through the skin and into the body until the tip of the cannula and the catheter are positioned inside the lumen of the vein or artery. The cannula or hollow needle is then removed while leaving the flexible catheter in place, which thus forms a conduit for the infusion or withdrawal of fluids between the vessel and the exterior of the body. When multiple transfers of fluid into or out of a vessel are required, the catheter can be left in place in order to reduce the need to reinsert the cannula or needle into the body, which is inconvenient and often painful for the patient.

One way to avoid repeated insertion of a needle into the vein is to use a venous or arterial cannula, which is usually a thin tube, the distal end of which is inserted through the skin into a blood vessel and maintained indwelling in the blood vessel with the proximal end of the tube being accessible on the skin surface. A trocar is used to puncture the vein and insert the silicon cannula that remains in the vein. Common complications associated with cannulation of a blood vessel include hematoma (accumulation and dotting of blood due to failure to puncture the blood vessel when the cannula is inserted), infiltration (delivery of an infusate to subcutaneous tissue instead of the vein), embolism (caused by air, a thrombus, or fragment of a catheter breaking off and entering the venous system), or phlebitis (inflammation of the vein resulting from mechanical or chemical irritation or from an infection). These complications are often more serious in children and elderly patients repeatedly exposed to cannula insertion and withdrawal.

In addition, in order to withdraw blood and because one cannot use a cannula that is already in a vein, one must repuncture again at least once a day for every hospitalized patient. That is the main difficulty and cause of hematomas and infections. Repeated punctures particularly in the elderly and in children, may cause the veins to collapse, making it difficult to find a suitable vein from which to withdraw blood.

### SUMMARY OF THE INVENTION

The objects of the inventions and disadvantages of prior devices are overcome by the present invention, which provides an improved cannula assembly, comprising a first cannula having a first length and first inner diameter, the first cannula having at least a flexible distal portion; a trochar having a second length and second diameter, the trochar having a sharp distal position capable of puncturing through skin and one or more blood vessels of a patient and sized such that the trochar may be inserted within the first cannula and extend beyond the flexible distal portion thereof; and at least one flexible second cannula for insertion in the first cannula, each second flexible cannula sized to extend beyond the distal portion of the first cannula for a predetermined length.

The invention further provides a method of inserting a cannula into a blood vessel to deliver a fluid to or withdraw a fluid from a blood vessel or other body cavity comprising providing a cannula assembly having a first cannula having a first length and a first inner diameter, the first cannula having at least a flexible distal portion, a trochar having a second length and a second diameter, the trochar having a sharp distal end capable of puncturing through skin and one or more blood vessels of a patient, the trochar sized such that the trochar may be inserted within the first cannula and extend beyond the flexible distal portion thereof, and at least one flexible second cannula for insertion in the first cannula, each second cannula sized to extend beyond the distal portion of the first cannula for a predetermined length; inserting the trochar into the first cannula such that the sharp distal tip or end extends beyond the distal end of the first cannula; inserting the first cannula and the trochar into a blood vessel of a patient by puncturing through skin into the blood vessel of the patient; withdrawing trochar from the first cannula; and inserting the second cannula into the first cannula such that the distal end of the second cannula extends predetermined distance in the blood vessel of the patient beyond the distal end of the first cannula.

The original cannula already has the sharp metal trocar and is used to puncture the skin and serve as a guide for the cannula. The first cannula remains in place, and another second cannula is inserted each time another blood sample is needed or desired. This saves the patient from multiple punctures and the system, may be sized for use with adult, pediatric, neonatal or elderly patients.

The system of the invention may be used, for example, for collecting a blood sample from a blood vessel, or infusing a liquid such as an intravenous fluid into a blood vessel. The system of the invention is particularly suited when repeated withdrawals of fluid are to be performed.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 shows an improved cannula for withdrawing fluid from a body cavity or delivering fluid to a body cavity, in accordance with one embodiment of the invention;

FIG. 2 shows the trocar of the system of FIG. 1 inserted into the cannula device of the FIG. 1;

FIG. 3a shows insertion of the tip of the trocar and cannula of the cannula assembly of the present invention into a blood vessel; and

FIG. 3b shows removal of blood from the blood vessel using the improved cannula assembly of FIG. 1.

### DETAILED DESCRIPTION OF THE PREFERRED EMBODIMENTS

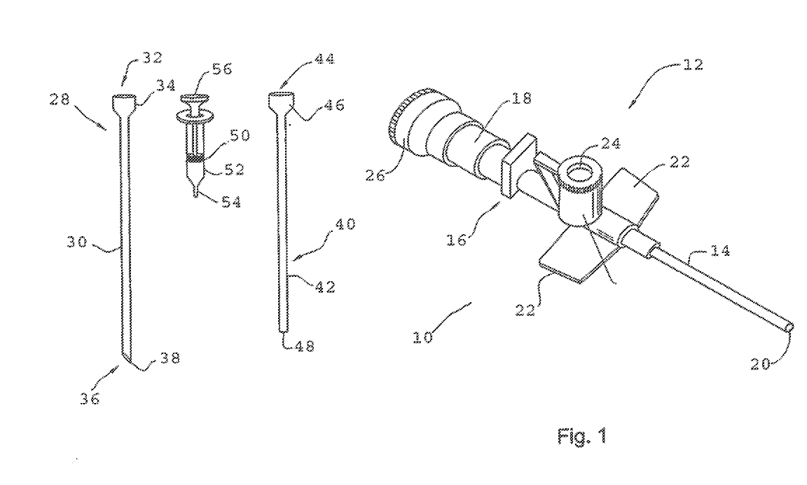

FIG. 1 shows a system 10 for transferring a fluid into or out of a body cavity such as an artery, vein, or other vessel (not shown), in accordance with one embodiment of the invention. The system 10 comprises a cannula device 12 having a flexible cannula 14 mounted onto a holder 16. The cannula device 12 also has a flashback chamber 18 that is used to determine when the distal end 20 of the cannula 14 is properly positioned inside a vessel by the presence of fluid such as blood in the flashback chamber 18, as explained below. Wings 22 on the holder 16 are used to insert the cannula device 12 into a blood vessel and can be adhered to the body surface using adhesive tape, for example (not shown). Fluids can enter or leave the cannula 12 either via a side port 24 or through a female Luer fitting 26, when a syringe or tubing (not shown) is attached to the Luer fining 26.

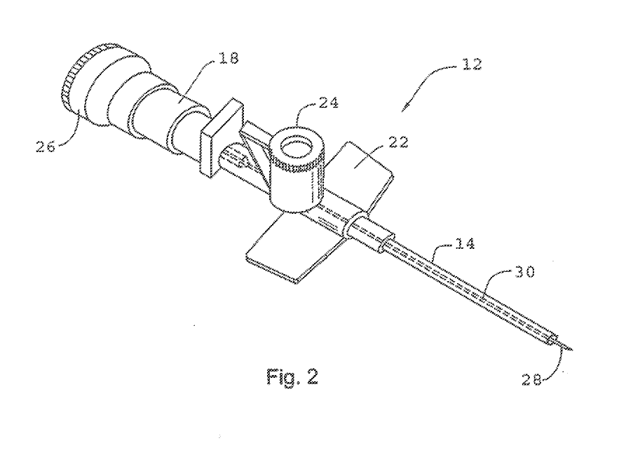

As shown in FIG. 2, the device 10 further comprises a trocar 28 having a slender, rigid, hollow tubular needle portion 30, a proximal end 32 that may be provided with a female Luer fitting 34 and a distal end terminating in a sharp pointed tip 38 suitable for puncturing skin and blood vessels.

The system 10 further comprises one or more flexible needles 40 having a flexible tubular portion 42, a proximal end 44 provided with a female Luer fitting 46, and a distal open end 48. The system 10 may also comprise a syringe 50 having a barrel 52 terminating in a male Luer fitting 54 configured to mate with the female Luer fitting 46 on the flexible needle 40, and a plunger 56. The flexible needle 40 may be integral with the syringe 50.

In use, the trocar 28 is inserted into the cannula device 12, as shown in FIG. 2. The outer diameter of the hollow tubular portion 30 of the trocar 28 is selected to be less than the inner diameter of the cannula 14, so that the trocar 28 is slidable within the cannula 14. The length of the hollow tubular portion 30 of the trocar 28 is selected to allow the tip 38 of the trocar 28 to extend beyond the tip 9 of the cannula 14 while the Luer fitting 26 of the trocar 28 is positioned in the Luer fitting 26 of the cannula device 14.

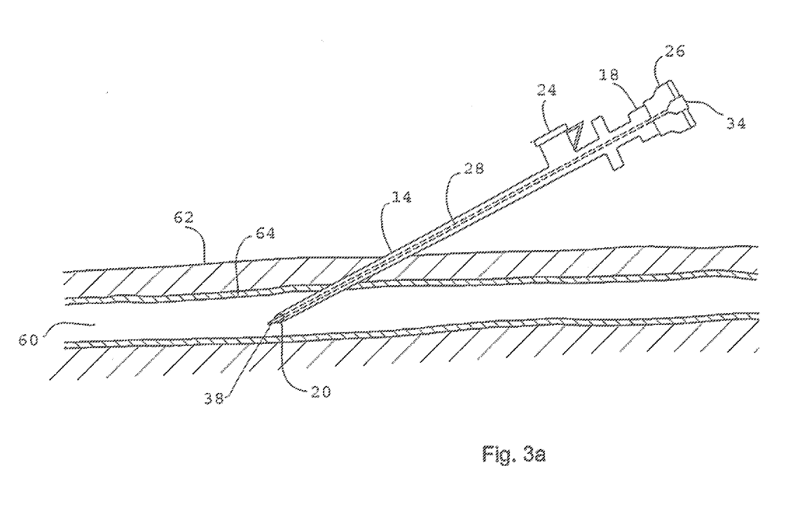

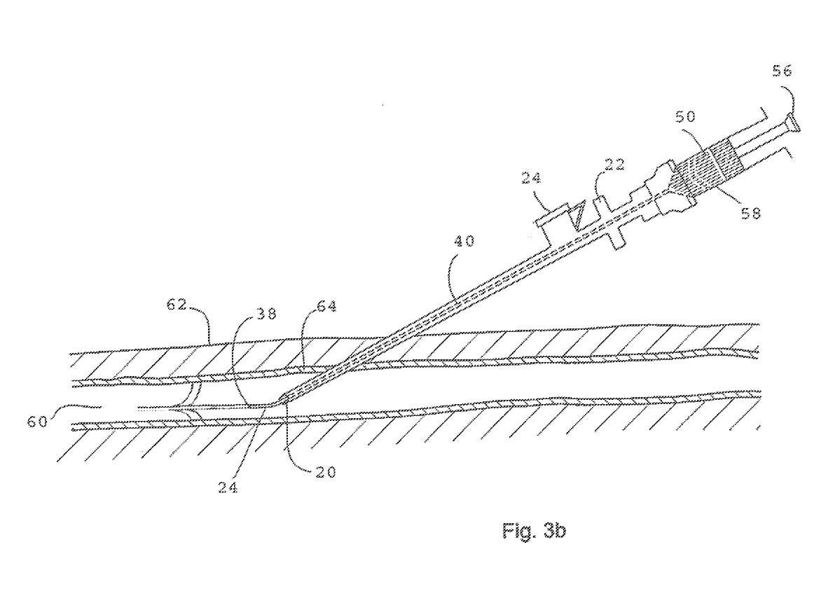

FIGS. 3a and b show use of the system 10 for obtaining a blood sample from a blood vessel 60. This is by way of example only and the system 10 may be used to deliver a fluid to any body cavity or to withdraw a fluid from a body cavity. As shown in FIG. 3a, after insertion of the trocar 28 into the cannula device 14, the tip 38 of the trocar 28 is used to pierce the skin surface 62. The tip 38 with the cannula 12 is then pushed forward until the tip 38 of the trocar 28 pierces the blood vessel wall 64 and the tip 38 of the trocar and the tip 29 of the cannula are positioned in the blood vessel 60. Insertion of the tip 29 of the cannula inside the blood vessel 60 is evidenced by the presence of blood in the flashback chamber 24. When the tip 20 of the cannula is positioned in the blood vessel the trocar 20 is removed from the cannula device 12. The cannula device 12 can then be affixed to the skin surface 62, for example, by taping the wings 22 to the skin surface 62. The cannula extends into a vein for a predetermined distance so as to avoid valves located in the vein and to avoid a vacuum or venturi effect, which is believed to be a problem with in-dwelling venous catheters. See FIG. 3b.

Now, as shown in FIG. 3b, the flexible needle 40 is mounted onto the syringe 50 with the plunger 56 in its depressed position. The flexible needle 40 is then inserted into the cannula device 12 until the tip 46 of the needle 40 extends beyond the tip 20 of the cannula 14 in the blood vessel 60. The plunger 56 can then be pulled back causing blood 58 to fill the barrel of the syringe 50.

After removal of blood, the syringe 50 and the flexible needle 40 are removed from the cannula device 12. The cannula device 12 remains affixed to the skin surface 62. The Luer fitting 26 is stoppered with a stopper (not shown) to prevent loss of blood from the blood vessel 64 or entry of pathogens into the device.

At any time when another blood sample 58 is to be withdrawn, a new flexible needle 40 is mounted onto a new syringe 50. The stopper (not shown) is removed and the flexible needle 40 is inserted through the cannula device 12 into the blood vessel 64, as shown in FIG. 3b, and the process repeated. Repeated withdrawals of blood 58 can thus be performed without piercing the skin repeatedly with the trocar 20 and without the need to change the cannula device 12.

It should be apparent to a person of ordinary skill in the art that the improved cannula assembly of the present invention provides many advantages over prior systems. First, the use of insertable elongated flexible cannula that extends beyond the distal end of the assembly avoids collapse of blood vessels near the tip of the assembly by extending a sufficient distance beyond the tip to avoid any blood vessel narrowing that may be present. Additionally, the flexible cannula is disposable thereby ensuring sterility. Further, the flexible cannula can be used to infuse fluid or medication into a vessel or to withdraw fluid or blood samples For testing and analysis.

The invention has been described with reference to particular embodiments, it should be understood that these embodiments are merely illustrative of the principles and practice of the present invention, and that various modifications will be apparent to persons of ordinary skill in the art, and that it is therefore intended that all search modifications are intended to be included within the spirit and scope of the invention as defined by the following claims.

1. An improved cannula assembly, comprising:
cannula having a first length and first inner diameter, the first cannula having at least a flexible distal portion;
a trochar having a second length and second diameter, the trochar having a sharp distal position capable of puncturing through skin and into a blood vessel of a patient and sized such that the trochar may be inserted within the first cannula and extend beyond the distal portion thereof; and
at least one flexible second cannula for insertion in the first cannula, the second flexible cannula sized to extend beyond the distal portion of the first cannula for a predetermined length within the blood vessel of the patient.

2. An improved cannula according to claim 1, wherein the predetermined length of the second cannula is chosen to avoid collapse of a patient's blood vessel in which the cannula is inserted.

3. An improved cannula assembly according to claim 1, wherein the cannula has a proximal end sized to accept a syringe or an intravenous solution adapter.

4. An improved cannula assembly according to claim 1, wherein the assembly is sized for use in a pediatric patient.

5. An improved cannula assembly according to claim 1, wherein the assembly is sized for use in a neonatal patient.

6. A method for infusing a fluid into or withdrawing a fluid from a blood vessel of a patient, comprising:
providing a cannula assembly having a first cannula having a first length and a first inner diameter, the first cannula having at least a flexible distal portion, a trochar having a second length and a second diameter, the trochar having a sharp distal end capable of puncturing through skin and a blood vessel of a patient, the trochar sized to insert and slide within the first cannula and extend beyond the distal portion thereof, and at least one flexible second cannula for insertion in the first cannula, each second cannula sized to extend beyond the distal portion of the first cannula for a predetermined length;
inserting the trochar into the first cannula such that the sharp distal tip extends beyond the distal portion of the first cannula;
inserting the first cannula and the trochar into a blood vessel of a patient by puncturing through skin into the blood vessel of the patient;
withdrawing the trochar from the first cannula; and
inserting the second cannula into the first cannula such that the distal end of the second cannula extends a predetermined distance in the blood vessel of the patient beyond the distal end of the first cannula.

7. A method in accordance with claim 6, further comprising connecting a syringe to a proximal end of the first cannula and withdrawing blood from the patient.

8. A method in accordance with claim 6, further comprising connecting a liquid dispenser to a proximal end of the first cannula and infusing a liquid into the blood vessel of the patient.

9. A method in accordance with claim 6, wherein the second cannula is withdrawn from the first cannula after use, leaving the first cannula in the blood vessel of the patient and capable of accepting another second cannula for repeated access to the blood vessel.
